(19) United States
(12) Patent Application Publication
(10) Pub. No.: US 2018/0000319 A1
(43) Pub. Date: Jan. 4, 2018

(54) Apparatus For Video-Endoscopy

(71) Applicant: Karl Storz GmbH & Co. KG, Tuttlingen (DE)

(72) Inventors: Harry Rutschmann, Klettgau (DE); Pieter H.A. Bos, Talheim (DE); Andreas Schmal, Emmingen-Liptingen (DE); Dominik Beutner, Orsingen-Nenzingen (DE)

(21) Appl. No.: 15/637,255

(22) Filed: Jun. 29, 2017

(30) Foreign Application Priority Data
Jun. 30, 2016 (DE) 102016111960.1

(51) Int. Cl. A61B 1/00; H04N 5/232; G02B 23/24

(52) U.S. Cl. CPC A61B 1/00052; G02B 23/2484; H04N 5/23203

(57) ABSTRACT

An apparatus for video endoscopy, in particular for industrial video endoscopy, has a device part which has a housing and an electronics component arrangement which is arranged in the housing. The electronics component arrangement is arranged within a sealed-off region in the housing. A heat sink is arranged in the housing, the said heat sink being thermally coupled to the electronics component arrangement in order to absorb heat from the electronics component arrangement. A fan for generating an air flow of ambient air is arranged in the housing, the said air flow flowing out of the housing along the heat sink in order to discharge the heat from the heat sink, wherein the air flow does not come into contact with the electronics component arrangement.

### CROSSREFERENCES TO RELATED APPLICATIONS

This application claims priority from German patent application 10 2016 111 960.1, filed on Jun. 30, 2016. The entire content of this priority application is incorporated herein by reference.

### BACKGROUND OF THE INVENTION

The invention relates to an apparatus for video endoscopy. The present invention relates, in particular, to an apparatus of this kind for video endoscopy which is used in industrial applications. The apparatus is used, for example, for inspecting machines, motors, turbines, reactors, buildings and the like.

An apparatus for video endoscopy has an endoscope to which a camera is connected or an endoscope in which the camera is integrated into the endoscope, be it in the distal region of the endoscope shaft or at the proximal end of the endoscope shaft. Both in the case of an endoscope with a connected camera and also in the case of an endoscope with an integrated camera, the endoscope is connected to a device part, which serves, inter alia, to control and supply power to the camera, by means of a cable which has signal and power supply lines. In this case, the cable is connected to the device part by means of a plug part. The device part has an electronics component arrangement comprising the electronic components, and the plug part can also have an electronics component arrangement comprising electronic components.

The device part can also have a light source, it being possible for the light which is generated by the said light source to likewise be connected to the endoscope by means of a fibre-optic cable.

An apparatus for medical video endoscopy is sold under the trade name TELE PACK X LED by KARL STORZ GmbH & Co. KG, Tuttlingen, Germany. An apparatus for industrial video endoscopy is sold under the trade name TECHNO PACK by KARL STORZ GmbH & Co. KG, Tuttlingen, Germany.

Irrespective of whether the apparatus is an apparatus for medical video endoscopy or an apparatus for industrial video endoscopy, there is a problem in that the electronics component arrangement generates heat in the housing of the device part during operation. This heat is an undesired by-product which reduces the performance of the electronics components and shortens the service life of the electronics components. For this reason, a cooling system is provided in the housing of the device part in the case of the known apparatus, the said cooling system having a fan which, during operation, draws in ambient air from the area surrounding the housing and generates an air flow which washes around the electronics component arrangement and thereby absorbs the heat from the electronics component arrangement and discharges the said heat out of the housing of the device part to the surrounding area at another point.

However, cooling of the electronics component arrangement of the device part using ambient air which washes around the electronics component arrangement has proven to be problematical when the video endoscopy apparatus is operated in a harsh environment as is the case in industrial applications. In harsh environments of this kind, the ambient air is admixed, for example, with dust, moisture or salt-containing substances. If the electronics component arrangement is cooled with ambient air, the electronic components can therefore be soiled, corrode and as a result of this malfunction. Therefore, the cooling concept of the known video endoscopy apparatuses is disadvantageous.

U.S. Pat. No. 8,767,060 B2 describes a video endoscopy apparatus in which various measures are provided in order to cool electronic components. According to one measure, the driver current is automatically reduced when an elevated temperature is detected. According to a further measure, heat sinks are arranged in the housing, which heat sinks form thermally conductive paths to the outside.

### SUMMARY OF THE INVENTION

The invention is based on the object of providing an apparatus for video endoscopy, in which apparatus the electronics component arrangement can also be cooled in harsh environments without being damaged by environmental influences.

The invention is further based on the object of providing an apparatus for video endoscopy which is compact in design.

The invention provides a video endoscopy apparatus, comprising a device part, the device part having a device part housing, a sealed-off region inside the device part housing, an electronics component arrangement arranged in the sealed-off region in the device part housing, a heat sink arranged in the housing, the heat sink being thermally coupled to the electronics component arrangement in order to absorb heat from the electronics component arrangement, a fan for generating an air flow of ambient air, the fan being arranged in the device part housing, the air flow flowing out of the housing along the heat sink in order to discharge the heat from the heat sink, wherein the air flow does not come into contact with the electronics component arrangement.

In the video endoscopy apparatus according to the invention, the electronics component arrangement is tightly encapsulated from its surrounding area. The tight encapsulation of the electronics component arrangement is realized by the region in the housing in which the electronics component arrangement is arranged being sealed off. Tight encapsulation of the electronics component arrangement can be realized by any suitable means, for example seals. Here, “tightly encapsulated” is intended to be understood to mean that the electronics component arrangement is protected, in particular, against contact with liquids, dust or other media which are harmful to the electronics component arrangement. The electronics component arrangement can be tightly encapsulated as a whole, component group by component group or component by component. Furthermore, a heat sink which is thermally coupled to the electronics component arrangement is arranged in the housing. The heat sink absorbs heat from the electronics component arrangement, which generates the said heat as an undesired by-product, during operation. The thermal coupling of the heat sink to the electronics component arrangement can be realized by the electronics component arrangement being at least partially in contact with the heat sink. Finally, a fan for generating an air flow of ambient air is arranged in the housing, wherein the air flow flows out of the housing along the heat sink in order to discharge the heat from the heat sink. In this case, the air flow does not come into contact with the electronics component arrangement. Therefore, ambient air can further be used for cooling purposes even in harsh environments, wherein, however, the ambient air comes into contact only with such parts within the housing, in particular with the heat sink or with the fan, which, however, are not sensitive to substances which are carried along by the ambient air. The video endoscopy apparatus according to the invention can therefore be used, in particular, in harsh environments, and is therefore suitable, in particular, for industrial video endoscopy.

The heat sink is preferably thermally coupled to the electronics component arrangement in the sealed-off region.

The quantity of heat produced during operation is greatest in the sealed-off region, and particularly effective heat discharge from the electronics component arrangement is achieved with the above measure.

In a further preferred refinement, the video endoscopy apparatus further comprises a plug part which can be coupled to the device part.

In this case, the plug part can be designed such that it can be inserted into a socket on the housing of the device part, wherein the housing of the plug part is substantially completely accommodated in the socket in the plugged-in state.

In a particularly preferred alternative refinement, the housing of the device part has, however, a cutout, wherein the plug part has a housing which can be inserted into the cutout in the housing of the device part and, in the inserted state, completes the housing of the device part and remains visible from the outside.

One advantage of this refinement of plug part and device part housing is that the plug part can be designed such that it can contain further components in the interior of its housing, as is provided in further preferred refinements. By way of example, a light source for endoscopic illumination and/or a laser light source, for example for measurement purposes, can be arranged in the plug part. The plug part can contain electrical components for transmitting electrical energy from the device part to a connected video endoscope, and/or the plug part can contain electronic components for data transmission (control commands and video data) from the device part to the video endoscope and from the video endoscope to the device part. Secondly, the entire apparatus remains compact overall, in spite of a relatively large plug part, because the plug part forms a portion of the housing of the device part and, in the inserted state, completes the housing of the device part.

A contour of the cutout in the housing of the device part and an outer contour of the housing of the plug part are preferably complementary, so that the plug part can engage into the cutout in an interlocking form-fit manner. In this case, the housing of the plug part closes off the cutout in the housing of the device part in an at least dust-tight manner.

The cutout in the housing of the device part and the housing of the plug part preferably extend over at least half the width of the housing of the device part in the direction of a width dimension of the housing of the device part.

Therefore, in this refinement, a large region of the entire width of the device part is used for the structural dimensioning of the plug part, so that a sufficient amount of installation space for the components (light source(s), electrical and electronic components) specified above is available in the housing of the plug part.

In a further preferred refinement, the housing of the plug part has a large number of cooling fins.

This refinement is made possible on account of the housing of the plug part remaining visible from the outside in the state in which it is inserted into the cutout in the housing of the device part, so that this portion of the housing of the plug part which is visible from the outside can advantageously be used for discharging heat from the interior of the plug part, in particular if one or more light sources are integrated in the plug part. This heat discharge can be very efficient in respect of the dimensioning of the plug part.

As already mentioned above, the plug part preferably contains a light source.

The light source serves, for example, to generate light which is passed from the plug part, starting via the cable by way of which the video endoscope is connected to the apparatus, to the video endoscope from which the said light then exits in order to illuminate a working or observation area.

As an alternative or in addition, the plug part preferably contains a laser source, in particular for a measurement system, in particular for a multipoint measurement system. Therefore, in this refinement, a measurement light source for a measurement system, which measurement light source is advantageously located in the plug part, can also be advantageously provided by the apparatus according to the invention.

The above-described aspect, according to which the housing of the plug part forms a portion of the housing of the device part in the state in which it is connected to the device part, by the plug part having a housing which can be inserted into a cutout in the housing of the device part and, in the inserted state, completes the housing of the device part and remains visible from the outside, and also the further above-described aspects which relate to the above-described aspect are also considered to be an independent invention, without all the features of patent Claim 1.

Accordingly, according to a second aspect, the invention also discloses a video endoscopy apparatus, comprising a device part, the device part having a device part housing having a cutout, a sealed-off region inside the device part housing, an electronics component arrangement arranged in the sealed-off region in the device part housing, the apparatus further comprising a plug part configured to be coupled to the housing part, the plug part having a plug part housing configured to be inserted into the cutout in the device part housing, and, in the inserted state, to complete the device part housing and to remain visible from the outside.

In a further preferred refinement, the plug part has a further electronics component arrangement which is arranged in a sealed-off region, wherein the plug part has a further heat sink which is thermally coupled to the further electronics component arrangement in order to absorb heat from the further electronics component arrangement.

Therefore, a cooling system for the plug part is also provided in the apparatus according to the invention. The plug part is arranged at the end of a cable which is connected or can be connected to the video endoscope or the camera of the endoscope, wherein the plug part can be coupled to the device part. In the case of the plug part, it is also advantageously provided that the electronics component arrangement of the plug part is tightly encapsulated from its surrounding area and is thermally coupled to a further heat sink in order to absorb heat from the further electronics component arrangement. The tight encapsulation of the electronics component arrangement of the plug part can be realized by the plug being tightly encapsulated substantially as a whole.

In respect of the plug part, it is also preferred when the further heat sink is thermally coupled to the further electronics component arrangement in the sealed-off region.

Furthermore, the plug part and the device part are preferably arranged along one axis, wherein the heat sink and the further heat sink adjoin one another in the direction of the axis, and wherein the air flow, which is generated by the fan, also flows along the further heat sink in order to discharge the heat from the further heat sink, without coming into contact with the further electronics component arrangement.

This refinement is particularly advantageous because only one fan is required for generating the air flow for discharging the heat from the further heat sink of the plug part too, this advantageously reducing the structural expenditure for the apparatus according to the invention. In addition, a very compact arrangement is produced owing to the series arrangement of the heat sink of the device part and of the further heat sink of the plug part. The device part, including the plug part, can therefore be configured with a very compact construction, so that the apparatus according to the invention is suitable, in particular, as a mobile, hand-held apparatus.

In a further preferred refinement, the heat sink has at least one air channel which runs through the interior of the heat sink, the air flow which is generated by the fan passing through the said air channel.

Owing to this measure, the air flow which is generated by the fan is even more effectively decoupled from the electronics component arrangement of the device part since the air flow is also shielded from the electronics component arrangement by the heat sink itself. In addition, the air flow can absorb the heat from the heat sink more effectively since all of the air is in contact with the heat sink.

The same measure is also advantageous in respect of the further heat sink of the plug part which accordingly has at least one further air channel which runs through the interior of the second heat sink, the air flow which is generated by the fan passing through the said further air channel.

In this case, it is further preferred when the at least one air channel in the heat sink of the device part communicates with the at least one further air channel in the further heat sink of the plug part.

In this way, the air flow which is generated by the fan can be guided through the heat sink and the further heat sink along a common air path.

The fan is preferably arranged on a housing-side end of the device part.

In this arrangement, the fan can, for example through a ventilation grille which is present in the housing of the device part, draw in ambient air and allow the said ambient air to flow through the device part along the heat sink and, according to one of the abovementioned refinements, also along the further heat sink of the plug part.

In this case, it is further preferred when the plug part is arranged on a housing-side end of the device part which is averted from the fan.

As a result, the air flow which is generated by the fan is guided from a first housing side to the opposite housing side, on which the plug part is arranged, with a compact design, without a plurality of fans or other complicated measures being required for guiding the air flow.

Further advantages and features can be gathered from the following description and the appended drawing.

It goes without saying that the features mentioned above and those still to be explained below can be used not only in the respectively indicated combination, but also in other combinations or on their own, without departing from the scope of the present invention.

### BRIEF DESCRIPTION OF THE DRAWINGS

An exemplary embodiment of the invention is illustrated in the drawing and will be described in more detail below with reference to said drawing, in which:

FIG. 1 shows a video endoscopy apparatus comprising a device part and an endoscope;

FIG. 2 shows a section through the device part in FIG. 1 along the section line II shown in FIG. 1, wherein a plug part of the video endoscope apparatus is coupled to the device part;

FIG. 3 shows a video endoscope apparatus comprising a device part and a plug part according to a further exemplary embodiment, wherein the plug part is removed from the device part;

FIG. 4 shows the video endoscope apparatus according to FIG. 3, wherein the plug part is coupled to the device part;

FIG. 5 shows a view of the video endoscope apparatus in FIG. 4 from the rear; and

FIG. 6 shows a partially broken-off illustration of the video endoscope apparatus in FIGS. 3 to 5.

### DETAILED DESCRIPTION OF PREFERRED EMBODIMENTS

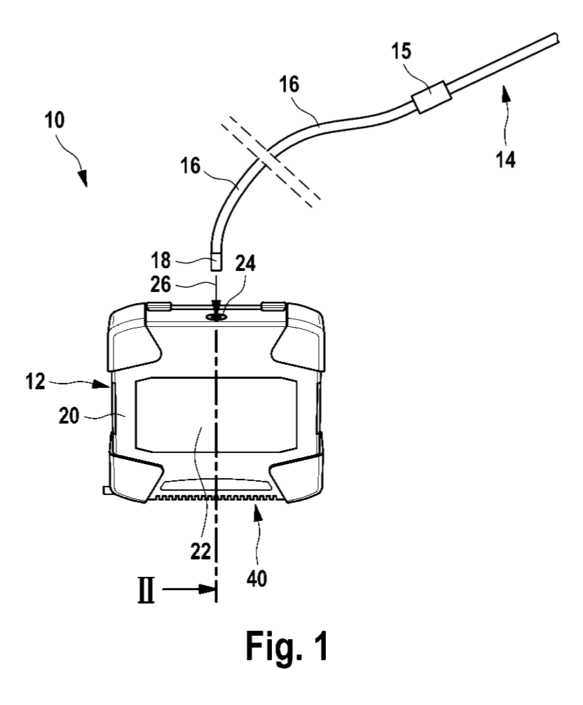

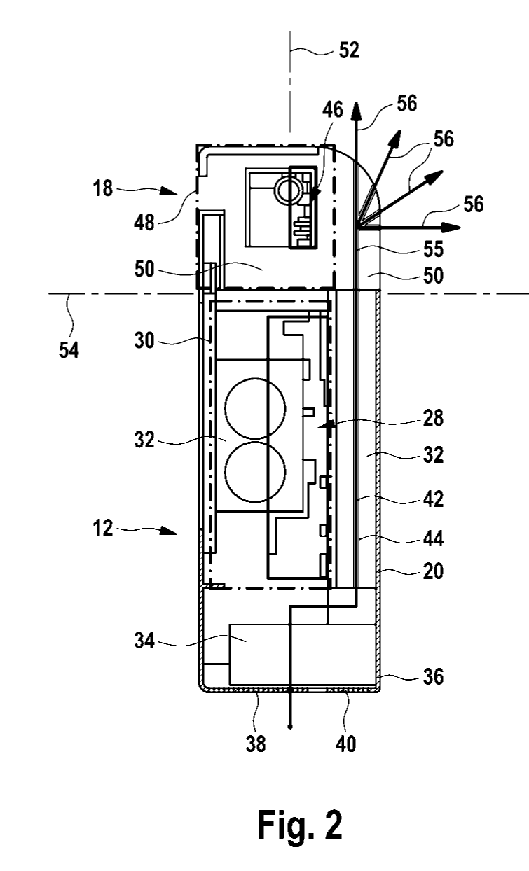

FIG. 1 shows an apparatus, provided with general reference symbol 10, for video endoscopy. Further details of the apparatus 10 are shown in FIG. 2.

The video endoscope apparatus 10 is used, in particular, in industrial applications for visual inspection. The video endoscope apparatus 10 is designed, in particular, to be used in harsh environmental conditions.

The video endoscope apparatus 10 has a device part 12. An endoscope 14 which is in the form of a video endoscope with an integrated camera or in the form of an endoscope with a connected camera, for example a camera 15, is also shown in FIG. 1. The endoscope 14 is connected or can be connected to a cable 16 which has a plug part 18 at the proximal end.

The device part 12 has a housing 20. A display screen or monitor 22 is arranged in the housing 20.

A plug socket 24 into which the plug part 18 can be inserted is further arranged on the housing 20, so that the plug part 18 can be coupled to the device part 12. This is illustrated by an arrow 26 in FIG. 1.

According to FIG. 2, an electronics component arrangement 28 which has one or more electronic components is arranged in the housing 20 of the device part 12. Since the device 12 primarily has the function of a camera control unit for the endoscope camera, the electronics component arrangement is, for example, part of the control electronics system for the endoscope camera.

During operation of the video endoscope apparatus 10, the electronics component arrangement 28 generates heat as an undesired by-product.

The electronics component arrangement 28 is tightly encapsulated from its surrounding area in the housing 20 of the device part 12, as is illustrated by a dash-dotted boundary line 30 which outlines a sealed-off region within the housing 20. The tight encapsulation of the electronics component arrangement 28 can be realized, for example, by one or more seal elements along the boundary line 30. The tight encapsulation of the electronics component arrangement 28 has the effect that the electronics component arrangement 28 does not come into contact with dust, liquids, spray mist and the like which can be carried along by the ambient air into the interior of the housing 20.

A heat sink 32 which is thermally coupled to the electronics component arrangement 28 in order to absorb heat from the electronics component arrangement 28 is arranged in the housing 20 of the device part 12. The thermal coupling between the heat sink 32 and the electronics component arrangement 28 can be realized by thermal radiation, thermal convection or by thermally conductive connection or by a plurality of these coupling types. In particular, the heat sink 32 is thermally coupled to the electronics component arrangement 28 in the sealed-off region 30.

Furthermore, a fan 34 is arranged in the housing 20 of the device part 12, specifically at a housing-side end 36 of the housing 20. Ventilation openings 38, for example in the form of a ventilation grille 40 (also see FIG. 1), are present at the end 36 of the housing 20. The fan 34 draws in ambient air via the ventilation openings 38 during operation. The fan 34 generates an air flow, which is indicated by a line 42 in FIG. 2, from the drawn-in ambient air.

The air flow 42 flows here through at least one air channel 44 which runs through the interior of the heat sink 32.

On account of the tight encapsulation of the electronics component arrangement 28, the air flow 42 does not come into contact with the electronics component arrangement 28 since the air flow 42 cannot enter the sealed-off region 30.

FIG. 2 shows the plug part 18 in the state in which it is coupled to the device part 12. The plug part 18 likewise has an electronics component arrangement 46 which has one or more electronic components. The electronics component arrangement 46 of the plug part 18 is tightly encapsulated from its surrounding area, as is illustrated by a dash-dotted boundary line 48 which outlines the sealed-off region of the plug part 18. The tight encapsulation of the plug part 18 can be realized by the plug part 18 being sealed off to the outside overall.

The plug part 18 has a heat sink 50 which is thermally coupled to the electronics component arrangement 46 in order to absorb heat from the electronics component arrangement 46. The thermal coupling between the heat sink 50 and the electronics component arrangement 46 can be realized, as described above for the heat sink 32 and the electronics component arrangement 28 of the device part 12, by thermal conduction, thermal convection and/or thermal radiation. Here, thermal coupling is also performed primarily in the sealed-off region 48 of the plug part 18 again.

As is clear from FIG. 2, the plug part 18 and the device part 12 are arranged along one axis 52, wherein the heat sink 50 of the plug part 18 and the heat sink 32 of the device part 12 adjoin one another in the direction of the axis 52, but separately from one another, as is indicated by a further axis 54 in FIG. 2. The axis 54 is the separating line between the plug part 18 and the device part 12.

The air flow which is generated by the fan 34 also flows along the heat sink 50 in order to dissipate the heat from the heat sink 50, without coming into contact with the electronics component arrangement 46, since the said electronics component arrangement is tightly encapsulated from its surrounding area. The heat sink 50, like the heat sink 32, has at least one air channel 55 which runs through the interior of the heat sink 50, the air flow which is generated by the fan 34 passing through the said air channel. The air flow passes out of the heat sink 50 into the surrounding area in accordance with flow arrows 56. Since the plug part 18 adjoins the device part 12 along the axis 53, and the fan 34 is arranged on the end 36 of the housing 20 of the device part 12 which is averted from the plug part 18, a compact design of the video endoscopy apparatus 10, in which only the one fan 34 is required in order to additionally also cool the plug part 18, is produced.

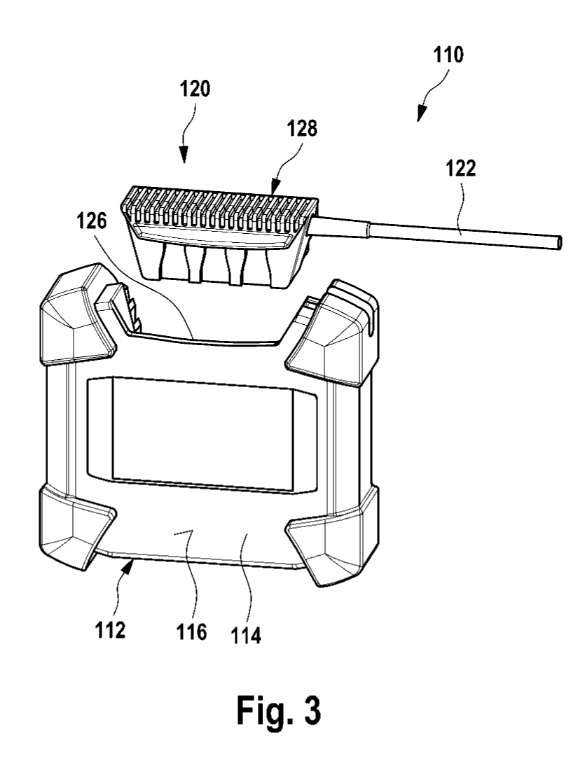

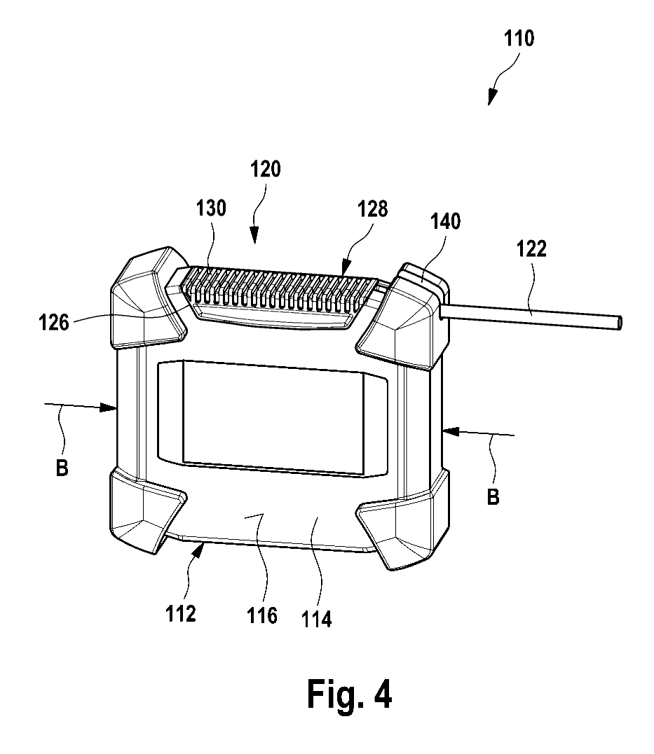

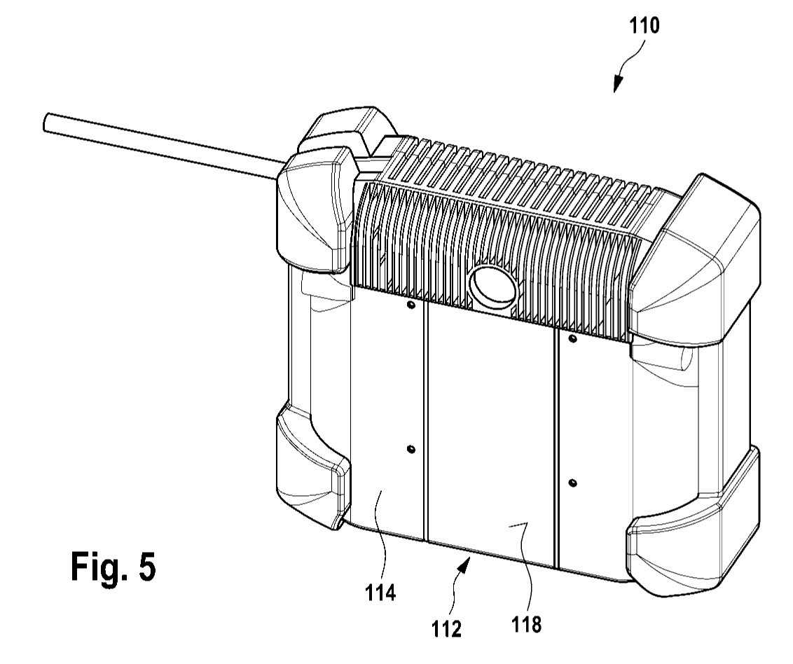

FIGS. 3 to 6 show a further exemplary embodiment of an apparatus, which is provided with the general reference symbol 110, for video endoscopy. The apparatus 110 has a device part 112 which has a housing 114. The housing 114 has a front side 116 which is shown in FIGS. 3 and 4, and a rear side 118 which is shown in FIG. 5.

The apparatus 110 has a plug part 120. The plug part 120 is connected or can be connected to a cable 122, wherein the cable 122 can be connected to an endoscope or video endoscope (not illustrated in FIGS. 3 to 6), for example to the endoscope 14 in FIG. 1.

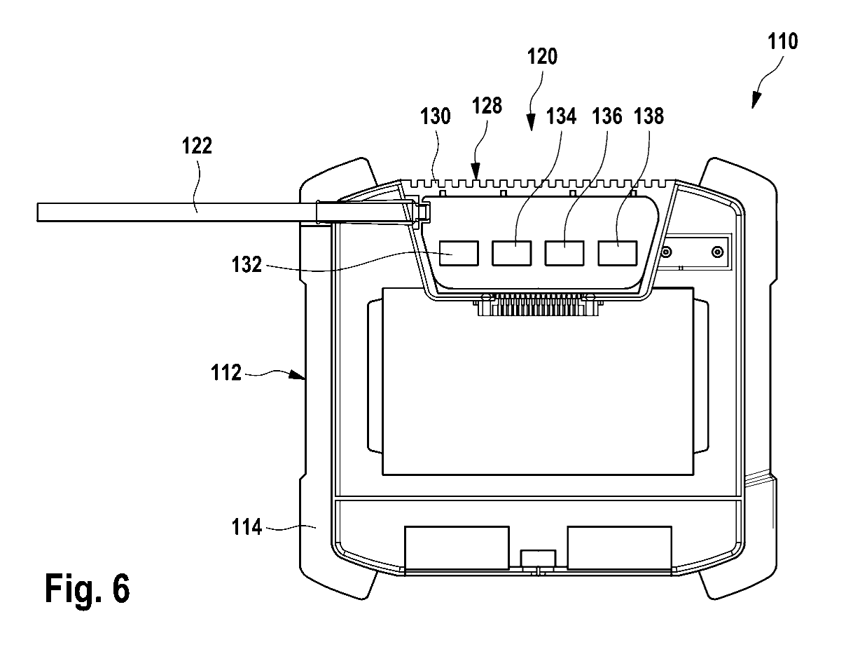

The plug part 120 can be coupled to, in particular can be plugged together with, the device part 112. FIG. 3 shows the plug part 120 in the state in which it is removed from the device part 112, while FIGS. 4 and 5 and also FIG. 6 show the plug part 120 in the state in which it is connected to the device part 112.

The housing 114 of the device part 112 has a cutout 126 into which the plug part 120 can be inserted, in particular can be inserted in an interlocking form-fit manner. To this end, the plug part 120 has a housing 128 which can be inserted into the cutout 126 in the housing 114 of the device part 112 and therefore completes and closes the housing 114 of the device part 112. In the state in which the plug part 120 is inserted into the cutout 126 in the housing 114, a portion of the housing 128 of the plug part 120 remains visible from the outside. In this case, the visible part of the housing 128 of the plug part 120 completes the housing 114 of the device part 112, in the exemplary embodiment shown the housing 128 of the plug part 120 completes the rest of the housing 114 of the device part 112 in a complementary manner. In other words, the housing 128 of the plug part 120 forms, in the inserted state of the plug part 120, a portion of the housing 114 of the device part 112. In the inserted state, the housing 128 closes off the cutout 126 at least in a dust-tight manner, preferably also in an air- and liquid-tight manner.

The housing 128 of the plug part 120 extends, in the direction of a width dimension of the housing 114 of the device part 112, which width dimension is illustrated by two arrows B in FIG. 4, at least over half of the said width dimension (cf. also the view from the rear in FIG. 5 in this respect).

That portion of the housing 128 of the plug part 120 which remains visible in the inserted state of the plug part 120 has a large number of cooling fins 130. These cooling fins 130 serve to discharge the heat from the interior of the plug part 120.

According to FIG. 6, different components, which are schematically illustrated in FIG. 6, are arranged in the interior of the plug part 120.

These components can include electrical components 132 which serve, for example, to transmit electrical energy from the device part 112 to the video endoscope (for example video endoscope 14 in FIG. 1). Furthermore, electronic components 134 which can serve for bidirectional data transmission, for example for transmitting control commands from the device part 112 to the video endoscope and/or for transmitting video data from the video endoscope to the device part 112, can be arranged in the plug part 120. In this case, electrical energy and/or data are/is transmitted via the cable 122.

Furthermore, a light source 136, preferably with a high light yield, is preferably arranged in the plug part 120, the light which is generated by the said light source likewise being transmitted via the cable 122 to the video endoscope to illuminate a working or observation area.

Furthermore, a laser source 138, which provides laser light for a measurement system, in particular a multipoint measurement system, can be arranged in the plug part 120.

The abovementioned components are able to be accommodated in the plug part 120 by the integration of the housing 128 into the housing 114 of the device part 112 as provided according to the invention, without the apparatus 110 as a whole having to be larger for this purpose.

Heat can advantageously be discharged via the cooling fins 130 not only from the interior of the plug part 120, but also from the interior of the device part 112. In terms of a cooling concept, the device part 112 can correspond substantially to the device part 12 of the apparatus 10, as has been described with reference to FIGS. 1 and 2.

Furthermore, a groove 140 into which the cable 122 is placed when the plug part 120 is connected to the device part 112 is made in the region of a corner of the housing 114 of the device part 112.

Otherwise, it goes without saying that the refinement of the plug part 120 and of the housing 114 with the cutout 126 can also be realized in the case of the apparatus 10 in FIGS. 1 and 2, wherein the cooling concept described with reference to FIG. 1 and the concept of sealing off electronics component arrangements can be retained.

What is claimed is:

1. A video endoscopy apparatus, comprising:
a device part, the device part having
a device part housing,
a sealed-off region inside the device part housing,
an electronics component arrangement arranged in the sealed-off region in the device part housing,
a heat sink arranged in the housing, the heat sink being thermally coupled to the electronics component arrangement in order to absorb heat from the electronics component arrangement,
a fan for generating an air flow of ambient air, the fan being arranged in the device part housing, the air flow flowing out of the housing along the heat sink in order to discharge the heat from the heat sink, wherein the air flow does not come into contact with the electronics component arrangement.

2. The video endoscopy apparatus according to claim 1, wherein the heat sink is thermally coupled to the electronics component arrangement in the sealed-off region.

3. The video endoscopy apparatus according to claim 1, further comprising a plug part having a plug part housing, the plug part being configured to be coupled to the device part.

4. The video endoscopy apparatus according to claim 3, wherein the device part housing has a cutout, and wherein the plug part housing is configured to be inserted into the cutout in the device part housing, and wherein at least a portion of the plug part housing, in the state in which it is inserted into the cutout, completes the device part housing and remains visible from the outside.

5. The video endoscopy apparatus according to claim 4, wherein the cutout and the plug part housing extend over at least half the width of the device part housing in direction of a width dimension of the device part housing.

6. The video endoscopy apparatus according to claim 3, wherein the plug part housing has a large number of cooling fins.

7. The video endoscopy apparatus according to claim 3, wherein the plug part contains a light source.

8. The video endoscopy apparatus according to claim 3, wherein the plug part contains a laser source.

9. The video endoscopy apparatus according to claim 3, wherein the plug part has a sealed-off region inside the plug part housing, and a further electronics component arrangement arranged in the sealed-off region, wherein the plug part has a further heat sink thermally coupled to the further electronics component arrangement in order to absorb heat from the further electronics component arrangement.

10. The video endoscopy apparatus according to claim 9, wherein the further heat sink is thermally coupled to the further electronics component arrangement in the sealed-off region.

11. The video endoscopy apparatus according to claim 9, wherein the plug part and the device part are arranged along a single common axis, wherein the heat sink and the further heat sink adjoin one another in the direction of the common axis, and wherein the air flow, which is generated by the fan, also flows along the further heat sink in order to discharge the heat from the further heat sink, without coming into contact with the further electronics component arrangement.

12. The video endoscopy apparatus according to claim 1, wherein the heat sink has at least one air channel running through the interior of the heat sink, the air flow generated by the fan passing through the air channel.

13. The video endoscopy apparatus according to claim 9, wherein the further heat sink has at least one further air channel running through the interior of the further heat sink, the air flow generated by the fan passing through the further air channel.

14. The video endoscopy apparatus according to claim 13, wherein the heat sink has at least one air channel running through the interior of the heat sink, the air flow generated by the fan passing through the air channel, wherein the at least one air channel communicates with the at least one further air channel.

15. The video endoscopy apparatus according to 1, wherein the fan is arranged on a device part housing-side end of the device part.

16. The video endoscopy apparatus according to claim 3, wherein the plug part is arranged on a device part housing-side end of the device part which is averted from the fan.

17. A video endoscopy apparatus, comprising:
a device part, the device part having
a device part housing having a cutout,
a sealed-off region inside the device part housing,
an electronics component arrangement arranged in the sealed-off region in the device part housing,
a plug part configured to be coupled to the device part housing, the plug part having
a plug part housing configured to be inserted into the cutout in the device part housing, and, in the inserted state, to complete the device part housing and to remain visible from the outside.

18. The video endoscopy apparatus according to claim 17, wherein the cutout in the device part housing has a first contour, and the plug part housing has a second contour, wherein the first and second contours are complementary with respect to each other, so that the plug part housing can engage into the cutout in an interlocking form-fit manner.

19. The video endoscopy apparatus according to claim 17, wherein the cutout and the plug part housing extend over at least half the width of the device part housing in direction of a width dimension of the device part housing.

20. The video endoscopy apparatus according to claim 17, wherein the plug part has a sealed-off region inside the plug part housing, and a further electronics component arrangement arranged in the sealed-off region.
